(19) United States
(12) Patent Application Publication
(10) Pub. No.: US 2013/0101623 A1
(43) Pub. Date: Apr. 25, 2013

(54) ATTENUATED MYCOBACTERIAL STRAIN AS NOVEL VACCINE AGAINST TUBERCULOSIS

(71) Applicant: UNIVERSITY OF MEDICINE AND DENTISTRY OF, Somerset, NJ (US)

(72) Inventors: Issar Smith, New York, NY (US); Rogelio Hernandez-Pando, Mexico, D.F. (MX); Riccardo Manganelli, Padova (IT)

(73) Assignee: UNIVERSITY OF MEDICINE AND DENTISTRY OF NEW JERSEY, Somerset, NJ (US)

(21) Appl. No.: 13/644,010

(22) Filed: Oct. 3, 2012

Related U.S. Application Data
(63) Continuation of application No. 13/282,929, filed on Oct. 27, 2011, now abandoned.
(60) Provisional application No. 61/407,154, filed on Oct. 27, 2010.

(51) Int. Cl. A61K 39/04

(52) U.S. Cl. CPC A61K 39/04
USPC 424/248.1; 435/253.1

(57) ABSTRACT

The present invention provides a novel attenuated vaccine for tuberculosis. Furthermore, when used as a subcutaneous vaccine, the present invention induces a higher level of protection than the current vaccine. Finally, the present invention results in less tissue damage and a lower number of colony forming units (CFU) in the lungs compared to subjects vaccinated with BCG.

### CROSS REFERENCE TO RELATED APPLICATIONS

This application claims the benefit under 35 U.S.C. Section 119(e) of U.S. Provisional Application Serial No. 61/407,154, filed Oct. 27, 2010, the entire disclosure of which is hereby incorporated by reference.

### FIELD OF THE INVENTION

The present invention relates to a novel and more protective vaccine for tuberculosis.

### BACKGROUND OF THE INVENTION

Tuberculosis (TB) is still one of the leading causes of mortality throughout the world. The HIV/AIDS pandemic, the deterioration in public health systems in developing countries, and the emergence of multi-drug resistance (MDR) forms of tuberculosis have contributed further to the pandemic. Prophylactic vaccination with the attenuated strain of Mycobacterium bovis Bacille Calmette-Guerin (BCG) is used in most countries. BCG vaccination, even if effective against severe forms of childhood tuberculosis, has a limited efficacy against adult pulmonary disease, the most transmissible form of the infection. Hence, new rationally constructed vaccine candidates are required.

Mycobacterium tuberculosis is a remarkable pathogen capable of adapting and surviving to various harsh conditions encountered during infection. Such adaptation is mostly due to a complex transcriptional regulatory network able to modulate the expression of its complex genome. Sigma factors bind to the RNA polymerase holoenzyme providing its specificity for particular promoters and play a key role in the regulation of gene expression and adaptation to stress in prokaryotes. The M. tuberculosis genome encodes for 13 sigma factors, 10 of which belong to the extracytoplasmic function (ECF) subclass (also referred to as group four). Among the mycobacterial sigma factors so far characterized, σ^{E} (belonging to the ECF subclass) is one whose involvement in virulence is very clear. A mutant in which its structural gene (sigE) was disrupted was not only sensitive to various surface disrupting stresses as the detergent sodium dodecyl sulphate (SDS), the cationic peptide polymyxin, and the antibiotic vancomycin, but was also unable to grow in resting macrophages, and dendritic cells, was more sensitive to killing from activated macrophages, and was severely attenuated in mice. The σ^{E} transcriptome was analyzed by DNA microarrays following SDS-induced surface stress and during macrophage infection: interestingly, σ^{E} was found to regulate genes involved in mycolic acid biosynthesis, and fatty acids degradation, as well as genes involved in membrane proteins quality control and membrane stabilization. Taken together, these data suggest that σ^{E} is responsible for controlling surface stability and composition following the exposure to damaging environmental conditions.

Recent in-vitro studies comparing the transcriptional response of human and murine macrophages, as well as human dendritic cells infected with wild type M. tuberculosis strain H37Rv and the sigE mutant, revealed that components of the σ^{E} regulon modulate the innate immune system, so that in the sigE mutant, there was an up-regulation of proteins of the acute phase response, Toll-like receptors 1 and 2, proinflammatory cytokines, chemokines and prostaglandins. Because the sigE mutant strain stimulates the host immune system during macrophage infection, the present invention involves this strain as an efficient live attenuated vaccine strain.

TB is still a very serious problem, especially in developing countries and populations at risk for multi-drug resistant forms of tuberculosis. Furthermore, the BCG vaccination, even if effective against severe forms of childhood tuberculosis, has a limited efficacy against adult pulmonary disease, the most transmissible form of the infection. Thus there remains a need for new rationally constructed vaccine candidates.

### SUMMARY OF THE INVENTION

The present invention is a Mycobacterium tuberculosis mutant which provides a novel attenuated vaccine. Furthermore, when used as a subcutaneous vaccine, the present invention induces a higher level of protection than the current vaccine. Finally, the present invention results in less tissue damage and a lower number of colony forming units (CFU) in the lungs compared to subjects vaccinated with BCG.

In a first embodiment, the present invention is a novel attenuated tuberculosis vaccine for humans.

In another embodiment, the present invention is a novel attenuated tuberculosis vaccine for livestock, especially cattle.

In yet another embodiment, the present invention provides a vector for delivery of protective antigens against other infectious diseases.

### BRIEF DESCRIPTION OF THE DRAWINGS

FIG. 1 depicts the pathogenicity of the sigE mutant after intratracheal inoculation;

FIG. 2 shows the quantitative expression of mRNA encoding cytokines determined by real time PCR in lungs from mice infected with sigE mutant, H37Rv or complemented strain;

FIG. 3 illustrates the lung bacilli load at the site of vaccination, inguinal lymph nodes, spleen, and lungs from BALB/c vaccinated with BCG or the sigE mutant at different time points before the challenge;

FIG. 4 is a quantification of IFN-γ by ELISA in cell suspension supernatants from inguinal lymph nodes, lungs and spleen after stimulation with culture filtrate mycobacterial antigens, and the immunodominant recombinant antigens ESAT-6 and Ag85, comparing BALB/c mice vaccinated with BCG and sigE mutant at different time points before the challenge;

FIG. 5 is a graphical representation of survival, lung bacilli loads, and histopathology after the intra-tracheal challenge with H37Rv or Beijing strain code 9501000 in BALB/c mice vaccinated with the sigE mutant or BCG, and compared to control non-vaccinated animals; and

FIG. 6 shows the virulence potential of each bacterial vaccine strain—BCG and sigE mutant, measured by the survival rates in two groups of subjects.

### DETAILED DESCRIPTION OF THE INVENTION AND PREFERRED EMBODIMENTS

The present invention is a novel attenuated vaccine for tuberculosis comprising a Mycobacterium tuberculosis mutant. It is as attenuated as BCG in immunodeficient nude mice, when inoculated subcutaneously. Furthermore, when used as a subcutaneous vaccine, the present invention was able to induce a higher level of protection than that of BCG, which is the vaccine currently used and the gold standard to evaluate new antitubercular vaccines at 2 and 4 months post intratracheal challenge with both H37Rv and a highly virulent strain of M. tuberculosis. The present invention also provides a more protective and less harmful vaccine, as mice vaccinated with the mutant showed less tissue damage and a lower number of CFU in the lungs compared to mice vaccinated with BCG.

Rationally attenuated, live replicating mutants of M. tuberculosis are potential vaccine candidates. The advantage of the present invention is that attenuated M. tuberculosis strains produce a large number of protective antigens, including those that are absent from BCG. Thus, vaccination with live attenuated M. tuberculosis can induce a stronger and longer immune stimulation, conferring higher levels of protection against TB than BCG. This invention, comprising the sigE mutant, is able to confer significantly better protection than BCG to challenge with virulent M. tuberculosis.

Thus, the present invention is a novel attenuated tuberculosis vaccine for humans or livestock. In another embodiment, the present invention provides a vector for delivery of protective antigens against other infectious diseases.

In yet another embodiment, the present invention is a double mutant, derived from the sigE mutant, wherein the vaccine is even more attenuated, highly immunogenic and over-expressive of protective antigens.

During infection, bacteria confront different environments determined by the site in which the pathogen resides and the activation of the host immune response. To survive and grow, the pathogen must be able to adapt to these different milieus. Most bacterial adaptive mechanisms are based on the regulation of gene expression, which consequently plays a very important role in bacterial pathogenesis. Examples of this regulation are the two-component regulatory systems like PhoP-PhoQ, and σ factors. σ factors of the σ⁷⁰ class are subdivided into four different groups depending on their sequence and function. σ^{E} is a member of the ECF subclass of sigma factors. It is induced after exposure to different stress conditions, such as heat shock, SDS-mediated cell surface stress, vancomycin, oxidative stress, alkaline pH, and during the growth in human macrophages. Its regulon includes several genes involved in stress response and surface biology, as mycolic acid biosynthesis, fatty acids degradation, membrane proteins quality control and membrane stabilization.

The sigE mutant is attenuated in immunodeficient SCID and immunocompetent BALB/c mice after intravenous infection. The present invention arose out of further characterization of the sigE mutant pathogenicity and immunogenicity in BALB/c mice after infection by the intratracheal route, and then evaluation of the potentiality of this mutant as an attenuated vaccine. The BALB/c mouse model of progressive pulmonary tuberculosis is suitable to determine the virulence and immune response induced by mutant mycobacteria, since it is based on aerogenic infection, which is the usual infection route in humans. Moreover, in this model the rate of bacterial multiplication in the lungs well correlates with the extent of tissue damage (pneumonia) and mortality, and the infection is successfully controlled as long as a strong Th1 cell response is sustained, which is endorsed by previous evidence on the protective role of Th1 cell-cytokines against mycobacterial infection.

Results confirmed that the sigE mutant is highly attenuated, permitting total survival of the animals after four months of infection, with significant lower bacilli loads and tissue damage than animals infected with the parental and complemented strains. In spite of the observation that lungs of mice infected with the sigE mutant had lower bacilli loads and inflammation, they exhibited significant higher expression of IFN-Γ and TNF-α than the lungs of mice infected with the parental or complemented strains suggesting that the sigE mutant elicits a stronger immune response. These results are in agreement with recent in-vitro observations of infected macrophages with the same mutant. These studies showed that in comparison with resting macrophages infected with the parental strain H37Rv, sigE mutant infected cells exhibited higher expression of the transcriptional factor T-bet and in consequence more IFN-γ production. Moreover, IFN-γ-activated macrophages infected in vitro with the mutant strain induced high expression of TNF-α, which could explain the reason of the high induction of iNOS expression that we detected in the sigE mutant infected lungs. Interestingly, the lungs of mice infected with the sigE mutant showed, during the late stage of infection, higher expression of IL-10, an antinflammatory cytokine that may limit migration of lymphocytes and reduce tissue damage. This finding was in perfect agreement with the high production of IL-10 that was previously determined in human dendritic cells infected in-vitro with the sigE mutant.

Another element of the present invention was based upon the increased expression of murine β-defensins 3 and 4 in the lungs of mice infected with the sigE mutant. These molecules are cationic natural antimicrobial peptides that can kill the microbes and some of them have chemotactic activities on immune cells. We have previously shown in this animal model after infection with H37Rv, a rapid and high expression of β-defensins 3 and 4 during the phase of efficient control of bacillary replication. This finding was in perfect agreement with the observation that macrophages infected in-vitro with the sigE mutant up-regulates genes encoding Toll-like receptors 1 and 2 and defensins. Thus, the predominant Th-1 response plus the high expression of β-defensins in mice infected with the sigE mutant could be the basis of its attenuation allowing the 100% survival in association with very low CFU and tissue damage.

These observations justify the experimental model used in the present invention, that the sigE mutant could have a strong potential as a novel attenuated vaccine, since the response to its infection fits well into the proposition that the aim of a “classical” vaccine is to mimic natural infection as closely as possible inducing a strong immune protective response without causing extensive disease. Moreover, the sigE mutant is a good vaccine candidate since it is highly attenuated in SCID mice infected by the aerosol and intravenous route, and produces a lower mortality than BCG when used to infect nude mice. Finally, another promising aspect of the present invention was that after vaccination and before challenge, spleen and lung cell suspensions stimulated with mycobacterial antigens from mice vaccinated with the sigE mutant were more efficient to produce IFN-y than those from animals vaccinated with BCG. Taken together these observations suggest that the present invention is safer and more immunogenic than BCG.

In addition to the down-regulation of the genes below its direct control, some of which are involved in surface biology, σ^{E} absence has a pleiotropic effect on the bacterial surface. This was demonstrated by the transcriptional profile of the sigE mutant after in-vitro macrophage infection, showing the induction of genes related with the cell wall structure, like rmlB2 which encodes for a putative galactose epimerase essential to the linking of peptidoglycan and mycolic acid, and tatA encoding one of the TAT system (twin arginine translocation) components, involved in the translocation of folded protein. Thus, the present invention most likely contains cell envelope defects resulting in both its attenuation and its high immunogenicity.

Other mycobacterial mutants have already been demonstrated to have good potential as new efficient vaccines. Three of them have been analyzed using the same experimental model that has led to the present invention: i) a mutant lacking phoP, which was able to induce similar protection than BCG (16); ii) a mutant lacking fadD26 (which lacks the cell wall lipid complex phthiocerol dimycocerosate), which conferred 70% survival after four months of challenge with the highly virulent strain Beijing 9501000, but showed only a partial attenuation ; iii) a mutant lacking the mammalian cell entry gene 2 (mce2), which was severely attenuated and induced a 72% survival after four months of challenge with the highly virulent strain Beijing 9501000. The present invention, employing the sigE mutant, is as attenuated as the mce2 mutant, but induced significantly better protection, allowing 80% mice survival after four months of challenge with strain Beijing 9001000. Thus, sigE mutant is until now the best vaccine candidate tested in this experimental mouse model. Similarly, for this purpose, a double mutant in order to create a more attenuated and highly immunogenic mutant or the over-expression of protective antigens in this strain could also be a viable vaccine candidate.

### EXAMPLES

The present invention is described more fully by way of the following non-limiting examples. Modifications of these examples will be apparent to those skilled in the art.

The following describes the materials and methods which led to the present invention.

Growth of bacterial strains

ST28 sigE mutant and its complemented derivative ST29 were obtained from M. tuberculosis H37Rv. The BCG strain used was M Bovis BCG Phipps. This BCG substrain was the most protective of 10 strains tested in our BALB/c model of progressive pulmonary tuberculosis. The Beijing strain code 9501000 was donated by Dr. D. van Soolingen (RIVM, The Netherlands). Strains were grown in Middlebrook 7H9 medium (Difco Laboratories) supplemented with OADC (Difco Laboratories). After 1 month of culture, mycobacteria were harvested, adjusted to 2.5×10⁵ bacteria in 100 μl phosphate buffered saline (PBS), aliquoted, and maintained at around 70° C. until used. Before use, bacteria were recounted and their viability checked.

Experimental model of progressive pulmonary tuberculosis in BALB/c mice

Virulence (as determined by survival, lung pathology and bacterial load) and immune response induced by each isolate were evaluated in 8 to 10 week old male BALB/c mice. To induce progressive pulmonary tuberculosis, mice were anaesthetized with sevoflurane and inoculated intratracheally with 2.5×10⁵ CFU of M. tuberculosis H37Rv, the sigE mutant or sigE complemented strain suspended in 100μl PBS. Infected mice were kept in a vertical position until the effect of anaesthesia passed. Animals were maintained in groups of five in cages fitted with microisolators connected to negative pressure. Twenty mice from each group were left undisturbed to record survival from day 8 up to day 120 after infection. Six animals from each group were sacrificed by exsanguination at 1, 3, 7, 14, 21, 28, 60 and 120 days after infection. One lung lobe, right or left, was perfused with 10% formaldehyde dissolved in PBS and prepared for histopathological studies. The other lobe was snap-frozen in liquid nitrogen then stored at 70° C. for microbiological and immunological analysis. All procedures were performed in a laminar flow cabinet in a biosafety level III facility. Animal work was performed in accordance with the Institutional Ethics Committee and the national regulations on Animal Care and Experimentation.

Preparation of lung tissue for histology and automated morphometry

One lobe of the lung was fixed by intratracheal perfusion with 10% formaldehyde for 24 hours, then sectioned through the hilus and embedded in paraffin. Sections, 5 μm thick, were stained with hematoxylin-eosin for the histological-morphometric analysis. The percentage of the pulmonary area affected by pneumonia was determined using an automated image analyzer (Q Win Leica, Milton Keynes).

Determination of colony-forming units (CFU) in infected lungs.

Right or left lungs from four mice at each time point, in two separate experiments, were used for colony counting. Lungs were homogenized with a Polytron (Kinematica, Luzern, Switzerland) in sterile 50 ml tubes containing 3 ml of isotonic saline. Four dilutions of each homogenate were spread onto duplicate plates containing Bacto Middlebrook 7H10 agar (Difco Labs, Detroit Mich., USA) enriched with oleic acid, albumin, catalase and dextrose. The time for incubation and colony counting was 21 days.

Real time PCR analysis of cytokines in lung homogenates

Left or right lung lobes from three different mice per group in two different experiments were used to isolate mRNA using the RNeasy Mini Kit (Qiagen), according to recommendations of the manufacturer. Quality and quantity of RNA were evaluated through spectrophotometry (260/280) and on agarose gels. Reverse transcription of the mRNA was performed using 5 μg RNA, oligo-dT, and the Omniscript kit (Qiagen, Inc). Real-time PCR was performed using the 7500 real time PCR system (Applied Biosystems, USA) and Quantitect SYBR Green Mastermix kit (Qiagen). Standard curves of quantified and diluted PCR product, as well as negative controls, were included in each PCR run. Specific primers were designed using the program Primer Express (Applied Biosystems, USA) for the following targets: glyceraldehyde-3-phosphate dehydrogenase (G3PDH): 5′-cattgtggaagggctcatga-3′, 5′-ggaaggccatgccagtgagc-3′, tumor necrosis factor alpha (TNF-α): 5′-tgtggcttcgacctctacctc-3′, 5′-gccgagaaaggctgcttg-3′, interferon gamma (IFN-γ): 5′-ggtgacatgaaaatcctgcag-3′, 5′-cctcaaacttggcaatactcatga-3′, interleukin 4 (IL-4): 5′cgtcctcacagcaacggaga 3′, 5′gcagcttatcgatgaatccagg 3′, interleukin 10 (IL-10): 5′aaaggcactgcacgacatagc3′, 5′tgcggagaacgtggaaaaac 3′, beta defensin 3 (mBD3): 5′tctgtttgcatttctcctggtg3′, 5′taaacttccaacagctggagtgg3′, beta defensin 4 (mBD4): 5′tctgtttgcatttctcctggtg3′ and 5′tttgctaaaagctgcaggtgg3′. See Table 1 below. Cycling conditions used were: initial denaturation at 95° C. for 15 minutes, followed by 40 cycles at 95° C. for 20 seconds, 60° C. for 20 seconds, 72° C. for 34 seconds. Quantities of the specific mRNA in the sample were measured according to the corresponding gene-specific standard. The mRNA copy number of each cytokine was related to one million copies of mRNA encoding the G3PDH gene.

Comparison of virulence attenuation and immunogenicity of BCG and sigE mutant vaccinated mice before the challenge

Experiments were conducted to confirm the attenuation of the sigE mutant in comparison with BCG in immunodeficient animals (nude mice), using the vaccination dose that conferred the best protection (8000 live bacilli, not shown). Groups of 20 nude mice were vaccinated subcutaneously at the base of the tail with one dose of 8000 live sigE mutant or BCG bacilli by the same route and the rate of survival was determined.

To study bacillary growth and ability of dissemination, bacilli colony forming units (CFU) were determined in different organs after subcutaneous vaccination. Groups of four BALB/c mice were killed at 15, 30 and 60 days post-vaccination. The inguinal lymph nodes, spleen, lungs, and the subcutaneous tissue at the site of vaccination (base of the tail) were immediately dissected and homogenized for determination of bacillary loads by CFU quantification following the same procedure described above.

Another group of four vaccinated BALB/c mice per time point was used to determine immunogenicity, by comparing the production of IFN-γ by cell suspensions from inguinal lymph nodes, spleen and lungs after stimulation with mycobacterial culture filtrate antigens (CFA), and the immunodominant recombinant antigens ESAT-6 and Ag85. After killing the mice, the spleen, inguinal lymph nodes and lungs were immediately removed and placed in 2 ml of RPMI medium containing 0.5 mg/ml collagenase type 2 (Worthington, N.J., USA), incubated for 1 hour at 37° C.; then, passed through a 70-μm cell sieve, crushed with a syringe plunger, and rinsed with the medium. Cells were centrifuged at 1500 rpm for 5 minutes and the supernatant was removed and red cells were eliminated with a lysis buffer. After washing, the cells were resuspended in RPMI medium supplemented with 2 mM L-glutamine, 100 U of penicillin per ml, 1 μg of streptomycin per ml (all from Sigma), and 10% fetal calf serum. Cultures for cytokine production (10⁶ cells in 1 ml of culture medium) were performed in flat-bottomed 24-well plates without and with mycobacterial antigens (CFA, ESAT-6, and Ag85). After 3 days of antigenic stimulation, the cells were centrifuged and the supernatant used for IFN-γ quantification through a commercial ELISA test kit (Pharmingen, San Diego, Calif., USA). Preliminary dose-response curve experiments showed that the best antigen concentration was 5 μg during 3 days of culture stimulation (data not shown).

Protection against M. tuberculosis H37Rv and high virulent Beijing-strain in BALB/c mice vaccinated with sigE mutants or BCG

Two separate experiments were performed using 10 mice for each of four experimental groups. Animals were vaccinated by inoculating the best protection dose of live bacilli (8000 cells, not shown) subcutaneously at the base of the tail. At 60 days post-vaccination, the first group of 10 mice was challenged through the intra-tracheal route with 2.5×10⁵ CFU of M. tuberculosis H37Rv, while the second group with the same number of animals was challenged by the same route and dose with the highly virulent Beijing-strain code 9501000. The third and fourth groups corresponded to control animals which were not vaccinated and were intra-tracheally infected with the same dose of either H37Rv or the Beijing strain. After 2 and 4 months post-challenge, levels of protection were determined by the quantification of CFU in lung homogenates, following the same procedure described above, and by automated morphometry, measuring the lung surface affected by pneumonia. Ten more animals per group were left untouched and deaths were recorded to construct survival curves.

Statistical analysis

Statistical analysis for survival curves was performed using Kaplan-Meier plots and Log Rank tests. Student's t-test was used to determine statistical significance of CFU, histopathology and cytokine expression, P<0.05 was considered as significant.

Results were obtained as described below and depicted graphically in the figures.

Characterization of the sigE mutant pathogenicity after intra-tracheal administration

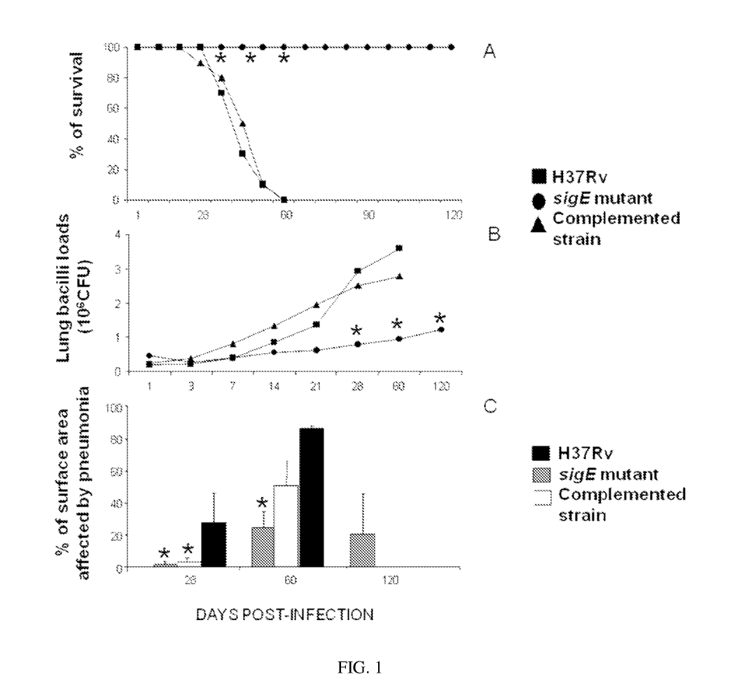

In order to characterize the sigE mutant attenuation in our model, groups of BALB/c mice (70 per group) were infected intratracheally with 2.5×10⁵ CFU of H37Rv, the sigE mutant, or its complemented strain. All of the animals infected with the sigE mutant survived after four months of infection. In contrast, mice inoculated with the complemented or parental strain started to die at three weeks post-infection and all had died by 8 weeks, as shown in FIG. 1A. These survival rates correlated well with the CFU in lung homogenates. During the first and second week of infection, similar numbers of CFU were detected in the three groups, whereas after days 21, 28, and 60 post-infection significantly lower bacterial loads were found in mice infected with the sigE mutant, compared to those detected in animals infected with the parental or complemented strains (see FIG. 1B). At day 120, animals infected with the mutant strain still showed a low bacterial burden.

The histopathological analysis showed progressive pneumonia produced after 28 days post-infection with M. tuberculosis H37Rv, reaching its peak at day 60 when 90% of the lung surface was affected. By contrast, in mice infected with the sigE mutant these pneumonic areas only involved 20% of the lung surface at day 60 and 120 post-infection.

FIG. 1 illustrates the pathogenicity of the sigE mutant after intratracheal inoculation. FIG. 1A represents the survival rates of BALB/c mice (20 mice per strain) infected by intratracheal injection of M. tuberculosis H37Rv, sigE mutant and complemented strain. On days 1, 3, 7, 14, 21, 28, 60 and 120 post-infection, mice were sacrificed and viable bacteria present in lungs were counted, yielding the results in FIG. 1B. The percentage of lung surface affected by pneumonia determined by automated morphometry is shown in FIG. 1C. (The results are expressed as the mean±standard deviations in four mice. Asterisks represent statistical significance (p>0.005) when compared to the H37Rv infected group.)

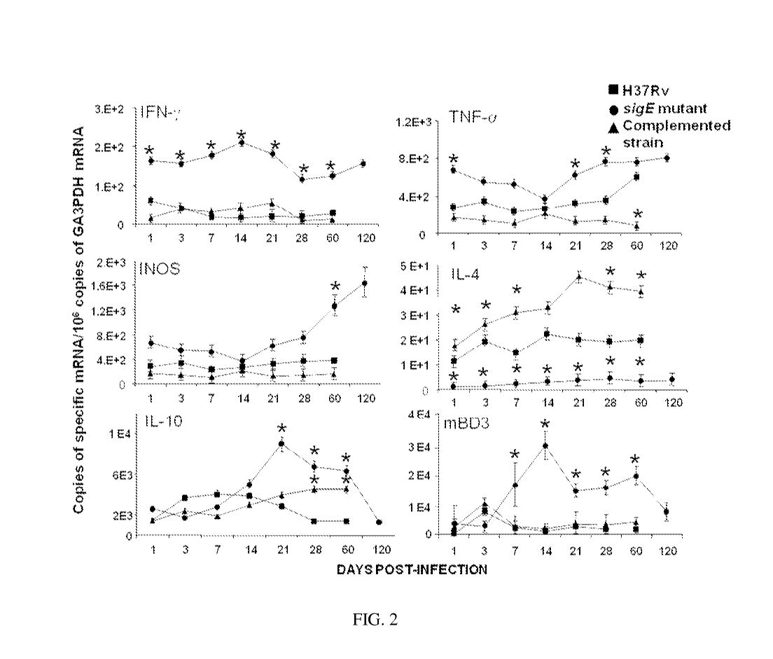

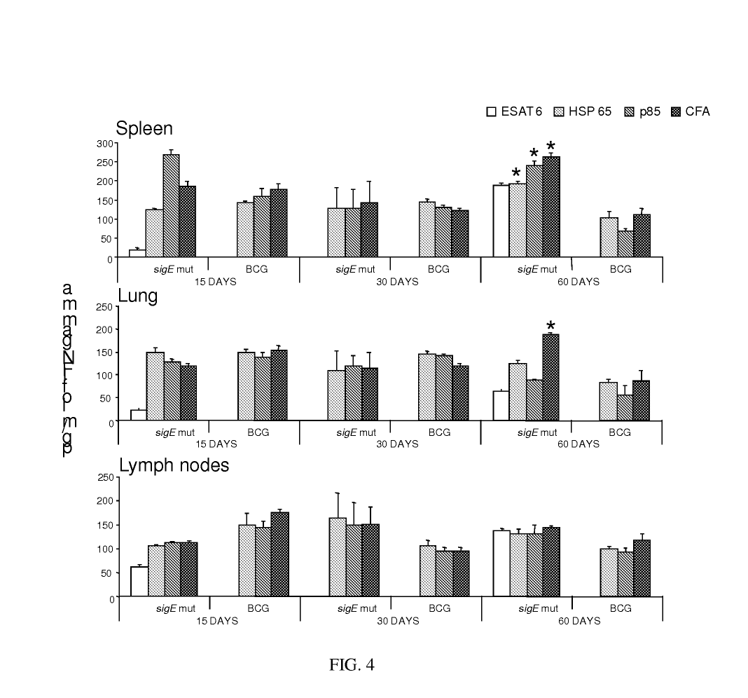

Although the lungs of mice infected with the sigE mutant showed significant lower bacilli loads and inflammation than animals infected with the parental or complemented strains, they showed a significant higher and constant expression of genes encoding IFN-y and TNF-a, as well as a progressive iNOS expression. These animals also showed constantly lower expression of IL-4 and a strikingly higher expression of β-defensins 3, as shown in FIGS. 2, and 4 (not shown) during the whole time of infection and a higher IL-10 expression during the late stage of infection.

FIG. 2 demonstrates the quantitative expression of mRNA encoding cytokines determined by real time PCR in lungs from mice infected with sigE mutant, H37Rv or complemented strain. Data are expressed as means and standard deviation from four different animals at each time point. Asterisks represent statistical significance (p<0.05) when compared with H37Rv infected mice. No data at day 120 post-infection is presented for H37Rv and parental strain infected mice because no surviving animals were available in these experiments.

Comparison of sigE mutant and BCG virulence and immunogenicity following vaccination

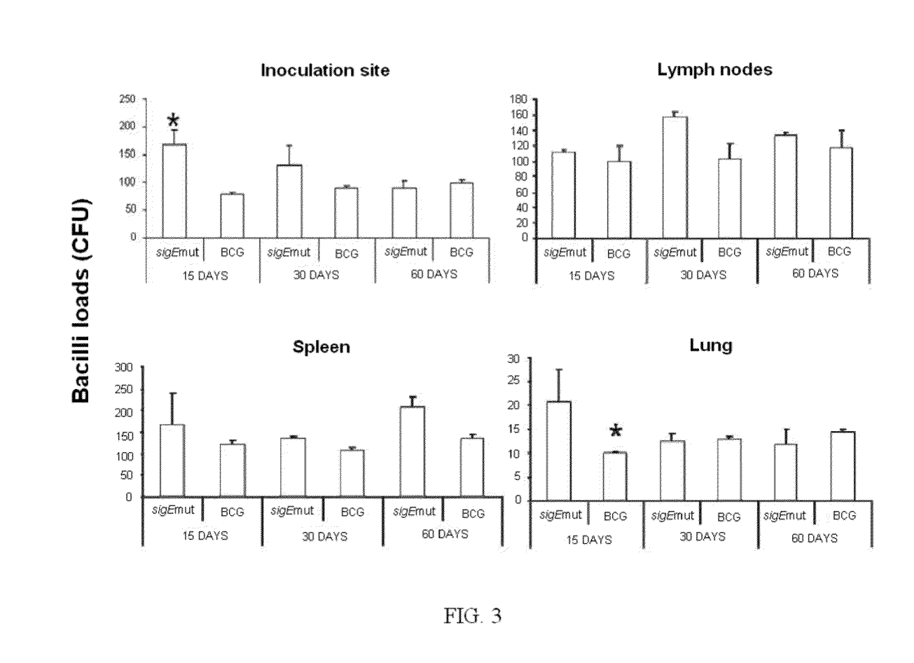

In order to compare the virulence of the sigE mutant to that of BCG, groups of BALB/c mice (12 per group) were inoculated subcutaneously with 8000 CFU of either of these two bacterial strains. Two weeks after vaccination, sigE mutant vaccinated animals showed a significant two-fold higher bacterial load at the site of vaccination and in the lungs. However, at days 30 and 60 post-vaccination, both groups of vaccinated animals showed similar bacilli loads in the inoculation site, inguinal lymph nodes, spleen and lungs, represented in FIG. 3, suggesting that the sigE mutant is not more virulent than BCG.

FIG. 3 depicts the lung bacilli load at the site of vaccination (in the subcutaneous tissue at the base of the tail), inguinal lymph nodes, spleen, and lungs from BALB/c vaccinated with BCG or the sigE mutant at different time points before the challenge. Bars represent the means and standard deviation from four different animals at each time point in two separate experiments. Asterisks represent statistical significance (p<0.05) among the indicated groups.

In order to compare the efficiency of cellular immunity activation induced by sigE mutant and BCG vaccination before challenge, spleen, lung and inguinal lymph node cell suspensions were collected and stimulated with mycobacterial antigens at different time points after vaccination, and the concentration of IFN-y in the supernatants was quantified through ELISA. FIG. 4 shows that spleen and lung cells from animals vaccinated with the sigE mutant stimulated with CFA or with the other recombinant antigens produced significant higher levels of IFN-γ than BCG-vaccinated mice at day 60 post-infection. Since BCG lacks the ESAT-6 structural gene, animals vaccinated with this strain did not produce or secrete significant amount of IFN-γ after stimulation with this antigen.

FIG. 4 indicates the results of the quantification of IFN-γ by ELISA in cell suspension supernatants from inguinal lymph nodes, lungs and spleen after stimulation with culture filtrate mycobacterial antigens (CFA), and the immunodominant recombinant antigens ESAT-6 and Ag85, comparing BALB/c mice vaccinated with BCG and sigE mutant at different time points before the challenge. Bars represent the means and standard deviation from four different animals at each time point. Asterisks represent statistical significance (p<0.05).

Comparative protection against M. tuberculosis H37Rv or Beijing-9501000 in BALB/c mice vaccinated with the sigE mutant or BCG

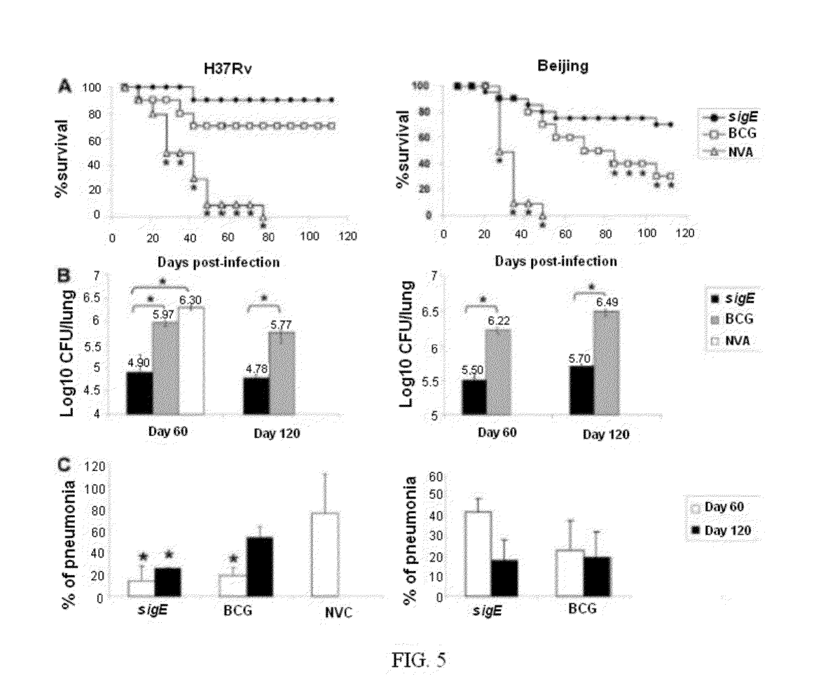

In order to compare the level of protection induced by BCG and the sigE mutant, groups of BALB/c mice (40 per group for 2 separate experiments) were vaccinated subcutaneously in the base of the tail with 8000 live bacilli of the sigE mutant or BCG. At 60 days post-vaccination, all mice were challenged intra-tracheally with 2.5×10⁵ M. tuberculosis H37Rv live bacilli. Ten mice were then euthanized at 60 or 120 days post-challenge. Levels of protection were determined by survival rates, quantification of CFU recovered from the lungs, and the extent of tissue damage by the evaluation of the percentage of the lung surface affected by pneumonia in both time points. After four months post-challenge, 98% of the mice vaccinated with the sigE mutant survived, while 20% of BCG vaccinated mice died. All control non-vaccinated animals died after 11 weeks of infection. These results correlated with lung bacilli loads and histopathology, showing significant less CFU and pneumonia in mice vaccinated with the sigE mutant than in BCG vaccinated or control non-vaccinated animals (see FIG. 5).

Survival, lung bacilli loads, and histopathology were quantified, shown in FIG. 5, after the intra-tracheal challenge with H37Rv (right panel) or Beijing strain code 9501000 (left panel) in BALB/c mice vaccinated with the sigE mutant or BCG, comparing with control non-vaccinated animals (NVA). Survival rates of vaccinated BALB/c mice (20 mice per strain) challenged with the indicated strain are shown in FIG. 5A. FIG. 5B shows the results at 2 (white bars) and 4 (black bars) months after challenge, when mice were sacrificed and viable bacteria present in lungs were counted. FIG. 5C illustrates the percentage of lung surface affected by pneumonia determined by automated morphometry after 2 (white bars) and 4 (black bars) months of intratracheal challenge. The results are expressed as the mean±standard deviations in four mice. Asterisks represent statistical significance (p>0.005) between the represented groups. No data at 4 months post-challenge is presented for the control non-vaccinated and BCG with the Beijing strain because no surviving animals were available.

In a second round of vaccination experiments, animals vaccinated following the same protocol were challenged with the highly virulent M. tuberculosis strain Beijing 9501000. Non-vaccinated animals started to die after four weeks after the challenge, and after 6 weeks were all dead, the results of which are shown FIG. 5. Mice vaccinated with BCG showed a 30% survival after 4 months post-challenge, whereas animals vaccinated with the sigE mutant exhibited a significant higher survival of 80%. These results were in agreement with lung CFU determinations, also presented in FIG. 5. Mice vaccinated with the sigE mutant showed three-fold fewer CFU in the lungs than BCG vaccinated mice and six-fold fewer bacilli loads than control non-vaccinated animals at day 60 after the intra-tracheal challenge with the Beijing strain (p<0.05). At day 120 after the challenge with the Beijing strain, sigE mutant vaccinated animals showed 50% more bacilli than at day 60 but three-fold fewer CFU than BCG vaccinated animals, as indicated in FIG. 5. BCG and sigE vaccinated mice showed similar percentage of lung surface affected by pneumonia, lower than in control non-vaccinated mice after 2 months from challenge (see FIG. 5).

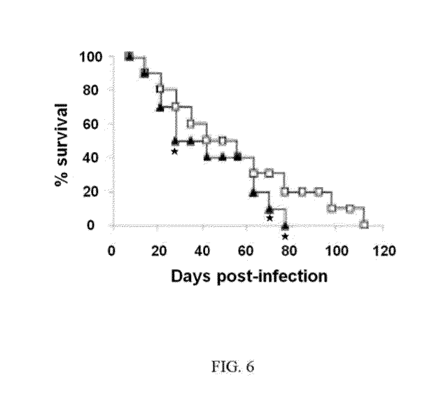

In order to further investigate the virulence potential of the sigE mutant, in contrast to that of BCG, two groups of twenty nude mice were inoculated subcutaneously with 8,000 CFU of either of the bacterial strains. As depicted in FIG. 6, results indicated that the sigE mutant is more attenuated than BCG in the immunodeficient subjects. Even if no significant difference existed between the two groups at the 50% survival time point, there was a significant difference in the rate of survival at the end of the experiment.

The foregoing examples and description of the preferred embodiments should be interpreted as illustrating, rather than as limiting the present invention as defined by the claims. All variations and combinations of the features above are intended to be within the scope of the following claims.

We claim:

1. A composition comprising a Mycobacterium tuberculosis mutant, for use as an attenuated vaccine.

2. The composition of claim 1 wherein the vaccine is administered to human beings.

3. The composition of claim 1 wherein the vaccine is administered to livestock.

4. A method of inoculation against infectious diseases, using the composition of claim 1 as a vector to deliver protective antigens.
